(19) United States
(12) Patent Application Publication
(10) Pub. No.: US 2003/0170883 A1
(43) Pub. Date: Sep. 11, 2003

(54) Microplate manufactured from a thermally conductive material and methods for making and using such microplates

(75) Inventors: Gregory R. Martin, Acton, ME (US); Paul M. Szlosek, Kennebunk, ME (US)

Correspondence Address: WILLIAM J. TUCKER, 8650 SOUTHWESTERN BLVD. #2825, DALLAS, TX, 75206, US

(73) Assignee: Corning Incorporated

(21) Appl. No.: 10/094,762

(22) Filed: Mar. 11, 2002

(51) Int. Cl. C12M 1/34

(52) U.S. Cl. 435/288.4; 435/305.2; 435/287.2

(57) ABSTRACT

A microplate that is manufactured from a thermally conductive material and methods for making and using the microplate are described herein. Basically, the microplate has a series of wells formed within a frame that is manufactured from a thermally conductive material which enables the wells to have relatively rigid walls which in turn makes it easier to handle the microplate. The thermally conductive material can be a metal or a mixture of a polymer (e.g., polypropylene, LCP) and one or more thermally conductive additives (e.g., carbon fiber, metal, ceramic). Also described herein is a tube manufactured from a thermally conductive material and methods for making and using the tube.

### BACKGROUND OF THE INVENTION

1. Field of the Invention

The present invention relates in general to the biotechnology field and, in particular, to a microplate manufactured from a thermally conductive material and methods for making and using such microplates.

2. Description of Related Art

Today polymerase chain reaction (PCR) processes which are associated with replicating genetic material such as DNA and RNA are carried out on a large scale in both industry and academia, so it is desirable to have an apparatus that allows the PCR process to be performed in an efficient and convenient fashion. Because they are relatively easy to handle and low in cost, microplates are often used during the PCR process. Reference is made to FIGS. 1A-1C, where there are illustrated different views of an exemplary traditional microplate 100 that is made from a polymeric material and has an array of conical or bullet shaped wells 102.

In accordance with the PCR process, a small quantity of genetic material and a solution of reactants are deposited within each well 102 of the traditional microplate 100. The traditional microplate 100 is then placed in a thermocycler which operates to cycle the temperature of the contents within the wells 102 (see FIG. 5 for an illustration of an exemplary thermocycler 500). In particular, the traditional microplate 100 is placed on a metal heating fixture in the thermocycler that is shaped to closely conform to the underside of the traditional microplate 100 and, in particular, to the exterior portion of the wells 102. A heated top plate of the thermocycler then tightly clamps the traditional microplate onto the metal heating fixture while the contents in the wells 102 of the traditional microplate 100 are repeatedly heated and cooled for around 90-150 minutes. Because, the traditional microplate 100 is made from a polymeric material which is a poor thermal conductor, the walls 104 of the wells 102 have to be molded as thin as possible so the thermocycler can effectively heat and cool the contents in the wells 102. The relatively thin well walls 104 in the traditional microplate 100 deform when they contact the metal heating fixture of the thermocycler to make good thermal contact. This requires that the traditional microplate 100 be made from a relatively non-rigid material such as polypropylene. Unfortunately, polypropylene tends to change dimensions when heated to relieve stress in the traditional microplate 100. As a result of the deformation of the relatively thin wells 102 and the tendency of the traditional microplate 100 to change dimensions during the thermal cycling, it is often difficult for a scientist to remove the traditional microplate 100 from the thermocycler. More specifically, as the number of wells 102 in the traditional microplate 102 increases from 96 wells to 384 wells to 1536 wells . . . , the force required to remove the traditional microplate 100 from the thermocycler also increases which further deforms the relatively thin, non-rigid, traditional microplate 100. The deformation of the relatively thin traditional microplate 100 is also undesirable because the contents in the wells 102 can be easily spilled which often requires that the wells 102 be sealed. Moreover, robotic handling systems have difficulty in handling the relatively thin traditional microplate 100 and removing the relatively thin traditional microplate 100 from the thermocycler. Accordingly, there is and has been a need for a microplate that does not suffer from the aforementioned shortcomings and other shortcomings of the traditional microplate 100. This need and other needs are satisfied by the microplate and the methods of the present invention.

### BRIEF DESCRIPTION OF THE INVENTION

The present invention includes a microplate manufactured from a thermally conductive material and methods for making and using the microplate. Basically, the microplate has a series of wells formed within a frame that is manufactured from a thermally conductive material which enables the wells to have relatively rigid walls which in turn makes it easier to handle the microplate. The thermally conductive material can be a metal or a mixture of a polymer (e.g., polypropylene, LCP) and one or more thermally conductive additives (e.g., carbon fiber, metal, ceramic). The present invention also includes a tube manufactured from a thermally conductive material and methods for making and using the tube.

### BRIEF DESCRIPTION OF THE DRAWINGS

A more complete understanding of the present invention may be had by reference to the following detailed description when taken in conjunction with the accompanying drawings wherein:

FIGS. 1A through 1C (PRIOR ART) respectively illustrate a perspective view, a cut-away partial perspective view and a cross-sectional side view of an exemplary traditional microplate 100;

FIGS. 2A through 2C respectively illustrate a perspective view, a cut-away cross-sectional top view and a cross-sectional side view of a microplate in accordance with a first embodiment of the present invention;

FIGS. 3A through 3C respectively illustrate a perspective view, a cut-away cross-sectional top view and a cross-sectional side view of a microplate in accordance with a second embodiment of the present invention;

FIGS. 4A through 4C respectively illustrate a perspective view, a cut-away partial perspective view and a cross-sectional top view of a microplate in accordance with a third embodiment of the present invention;

FIG. 5 is a perspective view of an exemplary thermocycler capable of heating and cooling the microplates shown in FIGS. 2-4;

FIG. 6 is a flowchart illustrating the steps of a preferred method for making the microplates shown in FIGS. 2-4 from a thermally conductive material that is a polymer mixed with one or more thermally conductive additives;

FIG. 7 is a flowchart illustrating the steps of another preferred method for making the microplates shown in FIGS. 2-4 from a thermally conductive material that is a metal;

FIG. 8 is a flowchart illustrating the steps of a preferred method for using the microplates shown in FIGS. 2-4;

FIGS. 9A through 9D respectively illustrate a perspective view and a cross-sectional top view of two different embodiments of a tube in accordance with the present invention;

FIG. 10 is a perspective view of an exemplary thermocycler capable of heating and cooling either of the tubes shown in FIG. 9;

FIG. 11 is a flowchart illustrating the steps of a preferred method for making the tubes shown in FIG. 9 from a thermally conductive material that is a polymer mixed with one or more thermally conductive additives;

FIG. 12 is a flowchart illustrating the steps of another preferred method for making the tubes shown in FIG. 9 from a thermally conductive material that is a metal; and

FIG. 13 is a flowchart illustrating the steps of a preferred method for using the tubes shown in FIG. 9.

### DETAILED DESCRIPTION OF THE DRAWINGS

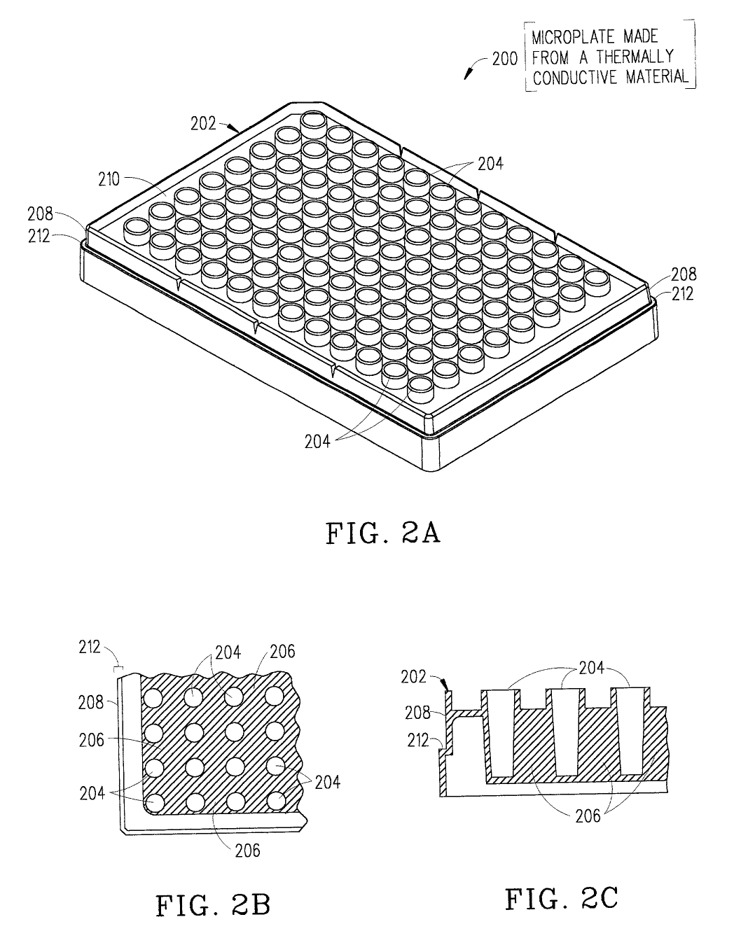

Referring to FIGS. 2-13, there are disclosed preferred embodiments of a microplate, a tube and preferred methods for making and using the microplate and the tube. Although the microplate and tube of the present invention are described below as being used in a PCR process, it should be understood that the microplate and tube can be used in a wide variety of processes.

Referring to FIGS. 2A through 2C, there are illustrated different views of a microplate 200 in accordance with a first embodiment of the present invention. The microplate 200 is manufactured from a thermally conductive material that is a polymer (e.g., polypropylene, LCP . . . ) mixed with one or more thermally conductive additives (e.g., carbon fiber, metals, ceramic . . . ). Or, the microplate 200 is manufactured from a thermally conductive material that is a metal (e.g., aluminum, zinc . . . ). A detailed discussion about the different types of thermally conductive materials that can be used to make microplate 200 and the other microplates 300 and 400 described below is provided after detailed discussions about the microplates 200, 300 and 400.

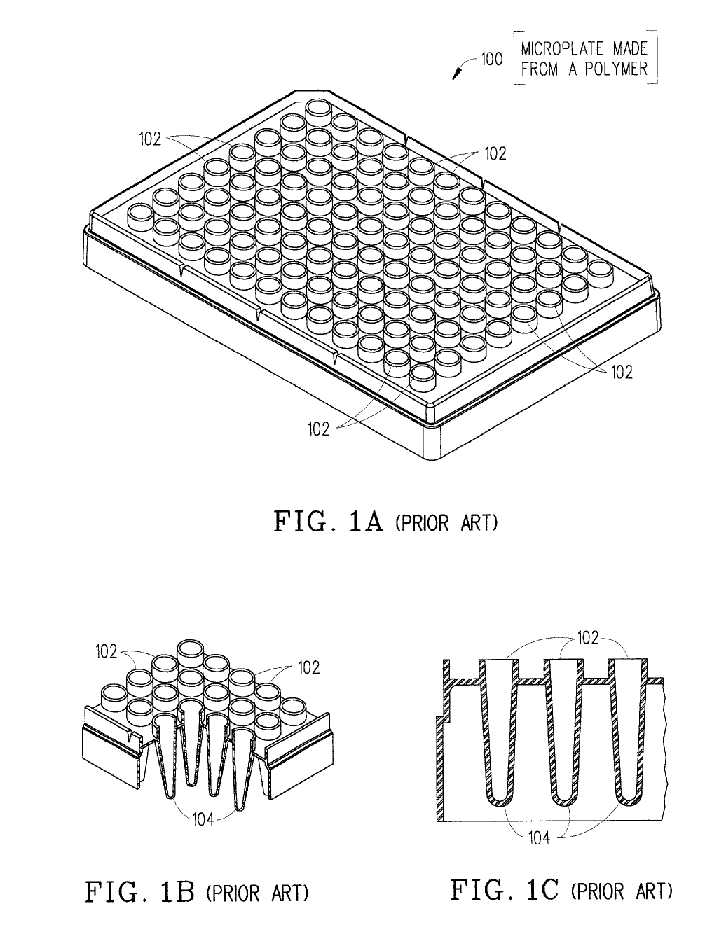

The microplate 200 being made from the thermally conductive material which is a “good” thermal conductor can dissipate heat/cold to the surrounding environment better than a similar sized traditional microplate 100 made from a polymer that is a “poor” thermal conductor. As such, the microplate 200 can be made thicker than the traditional microplate 100 and still function as well if not better than the thinner traditional microplate 100 (see FIG. 1). The thicker microplate 200 is more rigid and does not deform as much as the thinner traditional microplate 100 which makes it easier to handle than the traditional microplate 100. Again, one of the problems with the traditional microplate 100 is that it is relatively thin and as such it deforms when it is handled by a robotic handling system or it is inserted into or removed from a thermocycler.

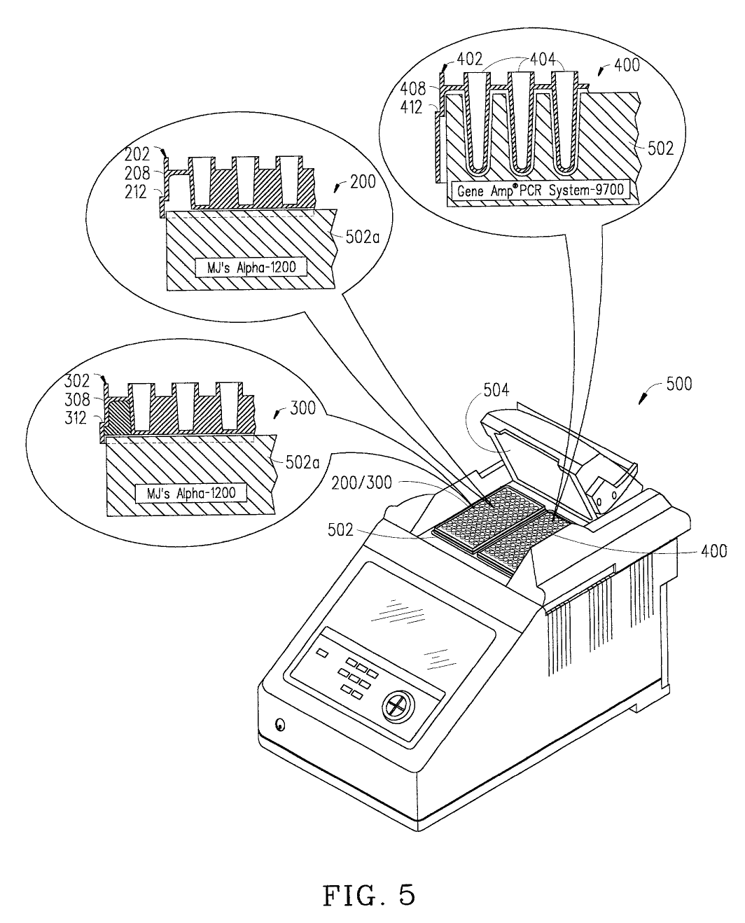

As illustrated, the microplate 200 includes a frame 202 that supports an array of ninety-six wells 204 where each well 204 shares a relatively thick wall 206 with adjacent wells 204. Please note the differences between the microplate 200 which has the relatively thick wall 206 that is shared between multiple wells 204 and the traditional microplate 100 which has relatively thin walls 104 that form each of the wells 102 shown in FIG. 1. The frame 202 which is shown as having a rectangular shape includes an outer wall 208 and a top planar surface 210 extending between the outer wall 208 and the wells 204. However, it should be understood that the frame 202 can be provided in any number of other geometrical shapes (e.g., triangular or square) depending on the desired arrangement of the wells 204. The outer wall 208 also has a rim 212 to accommodate the skirt of a microplate cover (not shown). The microplate 200 is configured to be placed within a thermocycler 500 which is described in greater detail below with respect to FIG. 5.

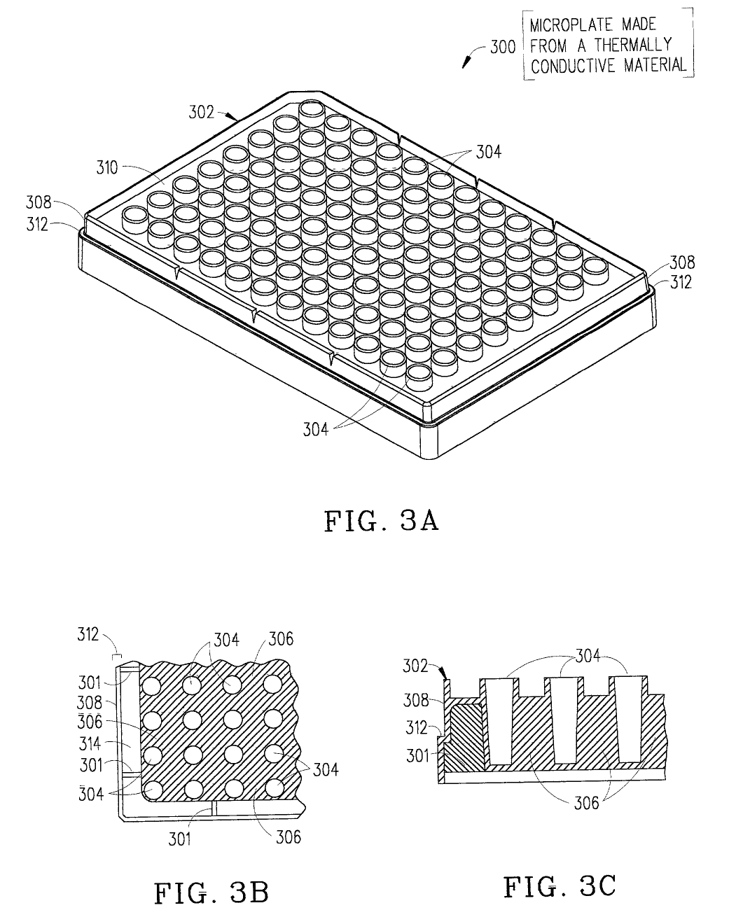

Referring to FIGS. 3A through 3C, there are illustrated different views of a microplate 300 in accordance with a second embodiment of the present invention. The microplate 300 has a configuration similar to microplate 200 except microplate 300 has one or more ribs 301 located between the bottoms of the wells 304 and the outer wall 308 (see FIGS. 3B and 3C). The ribs 301 help to support the outer wall 308 in a manner that makes the outer wall 308 more rigid so the microplate 300 can be easily handled by a robotic handling system.

As illustrated, the microplate 300 includes a frame 302 that supports an array of ninety-six wells 304 where each well 304 shares a relatively thick wall 306 with adjacent wells 304. Please note the differences between the microplate 300 which has the relatively thick wall 306 that is shared between multiple wells 304 and the traditional microplate 100 which has relatively thin walls 104 that form each of the wells 102 shown in FIG. 1. The frame 302 which is shown as having a rectangular shape includes an outer wall 308 and a top planar surface 310 extending between the outer wall 308 and the wells 304. However, it should be understood that the frame 302 can be provided in any number of other geometrical shapes (e.g., triangular or square) depending on the desired arrangement of the wells 304. The outer wall 308 also has a rim 312 to accommodate the skirt of a microplate cover (not shown).

As can be seen in FIGS. 3B and 3C, the microplate 300 also has a series of ribs 301 located between the bottoms of the wells 304 and the outer wall 308. The ribs 301 (three ribs 301 are shown in FIG. 3B) help to support the outer wall 308 in a manner that makes the outer wall 308 more rigid so the microplate 300 can be easily handled by a robotic handling system. Basically, the ribs 301 could be needed to support the outer wall 308 because the microplate 300 has an open area 314 between the wells 304 and the outer wall 308. The presence of the open area 314 helps to reduce the amount of thermally conductive material needed to make the microplate 300 which in turn saves money and reduces the weight of the microplate 300. It should be understood that the microplate 300 can have more ribs 301 than shown to help support the outer wall 308. Like microplate 200, microplate 300 is configured to be placed within a thermocycler 500 which is described in greater detail below with respect to FIG. 5.

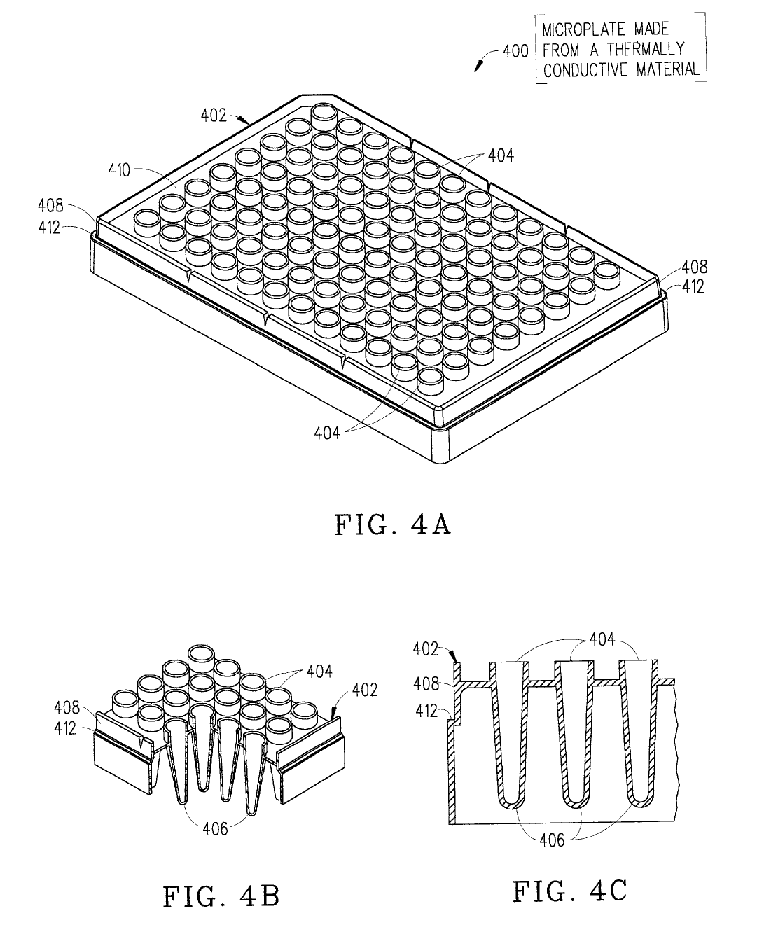

Referring to FIGS. 4A through 4C, there are illustrated different views of a microplate 400 in accordance with a third embodiment of the present invention. The microplate 400 has a configuration similar to the traditional microplate 100 except microplate 400 has a more rigid structure when compared to a similar sized traditional microplate 100 because the microplate 400 is made from a thermally conductive material. In particular, the thermally conductive material increases the mechanical properties (e.g., strength, stiffness . . . ) of the microplate 400, because the thermally conductive material is a “good” thermal conductor and can dissipate heat better than the polymer which is a “poor” thermal conductor that is used to make the traditional microplate 100. As a result, the microplate 400 does not distort as much as a similar sized traditional microplate 100. In other words, the traditional microplate 100 which is made form a polymer holds heat longer than the thermally conductive microplate 400 and as such has a tendency to deform more readily than microplate 400.

As illustrated, the microplate 400 includes a frame 402 that supports an array of ninety-six wells 404 each of which has a conical or bullet shape with relatively thin walls 406. The frame 402 which is rectangular in shape includes an outer wall 408 and a top planar surface 410 extending between the outer wall 408 and the wells 404. However, it should be understood that the frame 402 can be provided in any number of other geometrical shapes (e.g., triangular or square) depending on the desired arrangement of the wells 404. The outer wall 408 also has a rim 412 to accommodate the skirt of a microplate cover (not shown). Like microplate 200 and 300, the microplate 400 is configured to be placed within a thermocycler 500 which is described in greater detail below with respect to FIG. 5.

Although the microplates 200, 300 and 400 that have been described herein have ninety-six functional wells arranged in a grid having a plurality of rows and columns, it should be understood that the present invention is not limited to these arrangements. Instead, the present invention can be implemented in any type of microplate arrangement and can have any number of wells including 384 wells and 1536 wells.

Referring to FIG. 5, there is a perspective view of an exemplary thermocycler 500 capable of heating and cooling one or more microplates 200, 300 and 400. In accordance with the PCR process, a small quantity of genetic material and a solution of reactants are deposited within one or more wells 204, 304 and 404 of the microplate 200, 300 and 400. The microplate 200, 300 and 400 if need be is then covered by a microplate cover (not shown) or some other type of seal to help prevent the evaporation of the contents within the wells 204, 304 and 404. Thereafter, the microplate 200, 300 and 400 is placed in the thermocycler 500 which operates to cycle the temperature of the contents within the wells 204, 304 and 404.

As illustrated, microplate 200 and 300 is positioned onto a metal heating fixture 502a of the thermocycler 500 (e.g., MJ's Alpha-1200). The metal heating fixture 502a can be relatively flat to conform to the flat-bottomed wells 204 and 304 in the microplate 200 and 300 (see enlarged cross-sectional side views of the metal heating fixture 502a and microplates 200 and 300). Likewise, microplate 400 can be positioned onto a metal heating fixture 502b of the thermocycler 500 (e.g., GeneAmp® PCR System 9700). The metal heating fixture 502b can have a series of cavities that are shaped to closely conform to the exterior portion of the wells 404 in the microplate 400 (see enlarged cross-sectional side view of the metal heating fixture 502b and microplate 400). The thermocycler 500 also has a heated top plate 504 (shown in the open position) that tightly clamps the microplate 200, 300 and 400 onto the metal heating fixture 502a and 502b before the thermocycler 500 repeatedly heats and cools the contents within the microplate 200, 300 and 400. For instance, the thermocycler 500 can cycle the temperature of the contents within the wells 204, 304 and 404 from 95° C. to 55° C. to 72° C. some thirty times during the PCR process.

The use of a microplate 200, 300 and 400 that has a rigid structure makes it easy for a scientist or robot handling system to remove the microplate 200, 300 and 400 from the thermocycler 500 after completion of the PCR process. This is a marked improvement over the traditional microplate 100 that had a tendency to deform and stick to the metal heating fixture 502b of the thermocycler 500 which made it difficult for the scientist or robot handling system to remove the traditional microplate 100 from the thermocycler 500.

The microplate 200, 300 and 400 has a rigid structure because it is made from a thermally conductive material such as a polymer (e.g., polypropylene, LCP . . . ) mixed with one or more thermally conductive additives (e.g., carbon fiber, metals, ceramic (boron nitride) . . . ). Or, the microplate 200, 300 and 400 has a rigid structure because it is made from a thermally conductive material such as a metal (e.g., aluminum, zinc . . . ).

Described first is the microplate 200, 300 and 400 made from a thermally conductive material that is a polymer mixed with one or more thermally conductive additives. The polymer can be any type of thermoplastic. In experiments conducted by the inventors, it was easier for them to blend a thermally conductive material which had higher thermal conductivities (e.g., >5 W/mk) by mixing one or more thermally conductive additives with a crystalline polymer such as polypropylene or LCP (liquid crystal polymer) rather than with a noncrystalline polymer such as polycarbonate. However, it should be understood that both crystalline polymers and noncrystalline polymers can be made more thermally conductive with the addition of one or more thermally conductive additives. Also in the experiments, it was shown that microplate 200, 300 and 400 made from polypropylene or LCP that was blended with one or more thermally conductive additives did not inhibit the PCR process. Moreover, it has been shown that microplates 200, 300 and 400 made from polypropylene or LCP that were blended with one or more thermally conductive additives could be thermocycled in a manner such that they do not stress relieve at 100° C. and in a manner that their dimensions remain stable during the thermocycling.

The thermally conductive additives can be any material with a thermal conductivity greater than the base polymer. Below is a brief list of some exemplary thermally conductive additives including:

Carbon fibers and other graphitic materials some of which have thermal conductivities that are reportedly as high as 3000-6000 W/mk.

Metals including, for example, copper (400 W/mk) and aluminum (230 W/mk) that are micronized or flaked are preferred because of their high thermal conductivities.

Non-electrically conductive materials can also be used including, for example, crystalline silica (3.0 W/mk), aluminum oxide (42 W/mk), diamond (2000 W/mk), aluminum nitride (150-220 W/mk), crystalline boron nitride (1300 W/mk) and silicon carbide (85 W/mk).

It should be understood that the optimum concentration of the polymer relative to the amount of thermally conductive additive(s) depends on several factors including, for example, the type of polymer, the type of thermally conductive additive(s) and the desired thermal conductivity of the thermally conductive material.

As indicated above, there may be more than one thermally conductive additive added to the polymer to make the thermally conductive material. In fact, thermally conductive additives that have different shapes can be mixed together to contribute to an overall thermal conductivity that is higher than anyone of the individual additives alone would give. Moreover, an expensive thermally conductive additive (e.g., carbon fiber) can be mixed with a less expensive thermally conductive additive to reduce costs.

Today several types of commercially available thermally conductive materials which can be used to manufacture the microplate 200, 300 and 400. Four of these commercially available thermally conductive materials are briefly described below with reference to TABLES 1-4.

Table 1 illustrates some of the properties of a thermally conductive liquid crystalline polymer which is electrically non-conductive and sold by Cool Polymers Inc. under the product name of CoolPoly® D2:

TABLE 1
Thermal

| | | |
|---|---|---|
| Thermal Conducitivity | 15 W/m-K | ASTM E1461 |
| Thermal Diffusivity | 0.1 cm²/sec | ASTM E1461 |
| Heat Capacity | 0.9 J/g-° C. | ASTM E1461 |
| CLTE-parallel | 4 ppm/° C. | ISO 11359-2 |
| CLTE-normal | 10 ppm/° C. | ISO 11359-2 |
| Temp. of Deflection at 1.8 Mpa | 260° C. | ISO 75-1/-2 |
| UL Flammability | V0 at 1 | UL 94 mm |

Mechanical

| | | |
|---|---|---|
| Tensile Modulus | 21,000 MPa | ISO 527-1/-2 |
| Tensile Strength | 40 MPa | ISO 527-1/-2 |
| Izod-Unnotched | 3 ft-lbs/in | ASTM D4812 |
| Izod-Notched | 1 ft-lbs/in | ASTM D256 |

Electrical

| | | |
|---|---|---|
| Volume Resistivity | 10\hat{ }14 ohm · cm | IEC 60093 |

Physical

| | | |
|---|---|---|
| Density | 1.8 g/cc | ISO 1183 |
| Water Absorption | 0.1% | ISO 62 |

Table 2 illustrates some of the properties of a thermally conductive liquid crystalline polymer which is electrically conductive and sold by Cool Polymers Inc. under the product name of CoolPoly® E200:

TABLE 2
Thermal

| | | |
|---|---|---|
| Thermal Conducitivyt | 30 W/n-K | ASTM E1461 |
| Thermal Diffusivity | 0.2 cm²/sec | ASTM E1461 |
| Heat Capacity | 0.9 J/g-° C. | ASTM E1461 |
| CLTE-parallel | 5 ppm/° C. | ISO 11359-2 |
| CLTE-normal | 15 ppm/° C. | ISO 11359-2 |
| Temp. of Deflection at 1.8 Mpa | 260° C. | ISO 75-1/-2 |
| Temp. of Deflection at 0.45 Mpa | 270° C. | ISO 75-1/-2 |
| UL Flammability | V0 at 1 mm | UL 94 |

Mechanical

| | | |
|---|---|---|
| Tensile Modulus | 50000 MPa | ISO 527-1/-2 |
| Tensile Strength | 50 MPa | ISO 527-1/-2 |
| Nominal Strain at Break | 0.5% | ISO 527-1/-2 |
| Flexural Modulus | 49000 MPa | ISO 178 |
| Flexural Strength | 155 MPa | ISO 178 |
| Compressive Strength | 110 MPa | ISO 604 |
| Impact Strength-Charpy Unnotched | 5.5 kJ/m² | ISO 179 |
| Impact Strength-Charpy Notched | 3.5 kJ/m² | ISO 179 |

Electrical

| | | |
|---|---|---|
| Volume Resistivity | 500 ohm · cm | IEC 60093 |
| Surface Resistivity | 1 ohm/square | IEC 60093 |

Physical

| | | |
|---|---|---|
| Density | 1.76 g/cc | ISO 1183 |
| Water Absorption | 0.1% | ISO 62 |

Table 3 illustrates some of the properties of a thermally conductive liquid crystalline polymer which is electrically conductive, provides inherent EMI/RFI shielding and is sold by Cool Polymers Inc. under the product name of CoolPoly® E2:

TABLE 3
Thermal

| | | |
|---|---|---|
| Thermal Conductivity | 20 W/m-K | ASTM E1461 |
| Thermal Diffusivity | 0.1 cm²/sec | ASTM E1461 |
| Heat Capacity | 0.9 J/g-° C. | ASTM E1461 |
| CLTE-parallel | 7 ppm/° C. | ISO 11359-2 |
| CLTE-normal | 20 ppm/° C. | ISO 11359-2 |
| Temp. of Deflection at 1.8 Mpa | 260° C. | ISO 75-1/-2 |
| Temp. of Deflection at 0.45 Mpa | 270° C. | ISO 75-1/-2 |
| UL Flammability | V0 at 1 mm | UL 94 |

Mechanical

| | | |
|---|---|---|
| Tensile Modulus | 45000 MPa | ISO 527-1/-2 |
| Tensile Strength | 120 MPa | ISO 527-1/-2 |
| Nominal Strain at Break | 1.5% | ISO 527-1/-2 |
| Flexural Modulus | 35000 MPa | ISO 178 |
| Flexural Strength | 160 MPa | ISO 178 |
| Impact Strength-Charpy Unnotched | 5 kJ/m² | ISO 179 |
| Impact Strength-Charpy Notched | 2 kJ/m² | ISO 179 |

Electrical

| | | |
|---|---|---|
| Volume Resistivity | 0.1 ohm · cm | IEC 60093 |
| Surface Resistivity | 1 ohm/square | IEC 60093 |

Physical

| | | |
|---|---|---|
| Density | 1.7 g/cc | ISO 1183 |
| Water Absorption | 0.1% | ISO 62 |

Table 4 illustrates some of the properties of a thermally conductive liquid crystalline polymer which is electrically conductive and sold by RTP Company under the product name of RTP 3499-3 X 90363:

TABLE 4
Thermal

| | | |
|---|---|---|
| Thermal Conductivity, In-plane | 18 W/m-K | ASTM D3801 |
| Deflection Temperature @ 1.82 MPa | 260° C. | ASTM D648 |
| Flammability | V-0 @ 1.5 mm | ASTM D3801 |

Mechanical

| | | |
|---|---|---|
| Tensile Modulus | 58600 MPa | ASTM D638 |
| Tensile Strength | 75.8 MPa | ASTM D638 |
| Flexural Modulus | 41400 MPa | ASTM D790 |
| Flexural Strength | 137.9 MPa | ASTM D790 |
| Impact Strength, Unnotched 3.18 mm | 150 J/m | ASTM D256 |
| Impact Strength, Notched 3.18 mm | 32 J/m | ASTM D256 |

Electrical

| | | |
|---|---|---|
| Volume Resistivity | 10E-1 ohm · cm | ASTM D257 |
| Surface Resisitivity | 10E3 ohm/sq | ASTM D257 |

Compound Properties

| | | |
|---|---|---|
| Color | Natural | |
| Injection Pressure | 12000-18000 psi | |
| Injection Cylinder Temperature | 335-354° C. | |
| Mold Temperature | 66-121° C. | |
| Specific Gravity | 1.85 | ASTM D-792 |
| Molding Shrinkage | 0.05% | ASTM D-955 |

A test has been performed on a 384 style microplate 200 with 100 μL of water per well 204 and a thermocouple held in the middle of the water. The bottom of the microplate 200 was placed against a 100° C. hot plate so that heat was transferred from only one plate. The microplate 200 was made from a thermally conductive liquid crystalline polymer sold by Cool Polymers Inc. that had a thermal conductivity of 7 W/mk (not one of the commercially available products described above). In the test, the water in the wells 204 of microplate 200 was heated from 55° C. to 95° C. in 25 seconds. In contrast, an identical traditional microplate molded from polypropylene with a thermal conductivity of 0.3 W/mk had the same amount of water in the wells which was heated from 55° C. to 88° C. in 180 seconds.

As briefly described above, the microplate 200, 300 and 400 can also be made from a thermally conductive material that is a metal. In one embodiment, the microplate 200, 300 and 400 can be made in a machine from a metal by a process known as die casting. The metal can be zinc, aluminum, magnesium, copper and a wide variety of other metals. The microplate 200, 300 and 400 made from metal can be used as is or have the surface of the metal treated with a surface coating to keep the metal from contacting the PCR solution. For example, the microplate 200, 300 and 400 can be electroplated or electrolessly plated with a suitable metal, anodized (if the plate is made from aluminum or one of it's alloys), or coated with an organic barrier coating such as crosslinked acrylate, high temperature wax, etc . . .

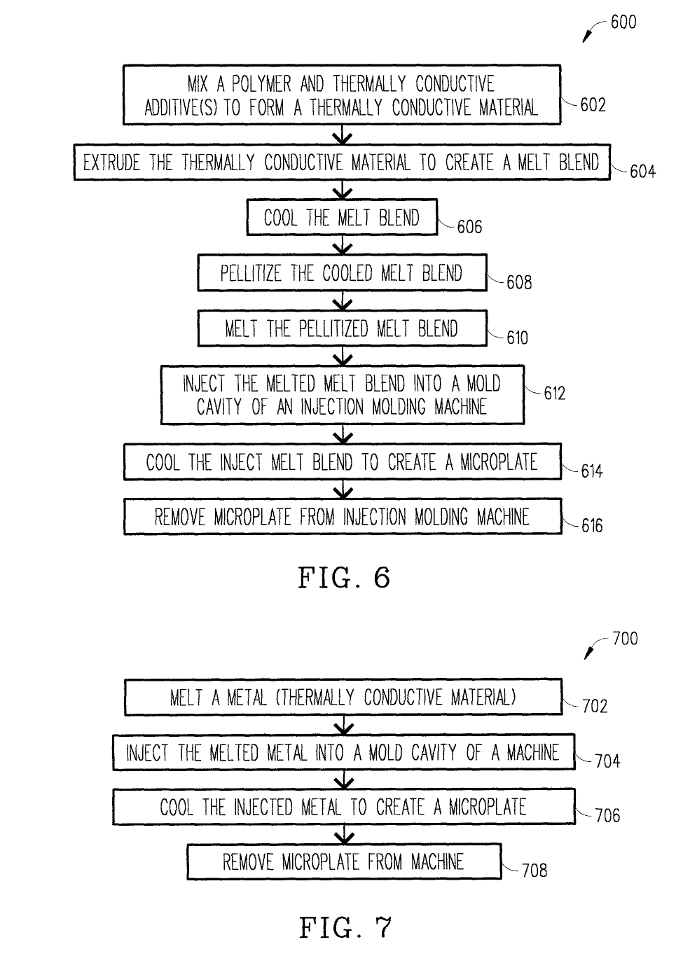

Referring to FIG. 6, there is a flowchart illustrating the steps of a preferred method 600 for making microplate 200, 300 and 400 using the thermally conductive material that is a polymer mixed with one or more thermally conductive additives. The microplate 200, 300 and 400 can be manufactured by mixing (step 602) a polymer (e.g., crystalline polymer) and one or more thermally conductive additives to form a thermally conductive material. In the preferred embodiment, the microplate 200, 300 and 400 is made from polymer such as polypropylene and a thermally conductive additive such as carbon fiber, metal, ceramic (boron nitride) . . .

The next step in manufacturing the microplate 200, 300 and 400 includes extruding (step 604) the polymer that is mixed with one or more thermally conductive additives to create a melt blend. In particular, the polymer and thermally conductive additive(s) can be fed into a twin-screw extruder with the help of a gravimetric feeder to create a well dispersed melt blend. The extruded melt blend is then run through a water bath and cooled (step 606) before being pelletized (step 608) and dried. The pelletized melt blend is heated and melted (step 610) by an injection molding machine which then injects (step 612) the melt blend into a mold cavity of the injection molding machine. The mold cavity includes sections shaped to form the microplate 200, 300 and 400. The injection molding machine then cools (step 614) the injected melt blend to create the microplate 200, 300 and 400. Finally, the microplate 200, 300 and 400 is removed (step 616) from the injection molding machine.

An advantage of the microplate 200, 300 and 400 made from a thermally conductive material is that the microplate 200, 300 and 400 is relatively rigid and as such can be easily removed from the mold cavity of the injection molding machine. This is a marked improvement over the state of the art where the traditional microplate 100 would warp and deform upon removal from the mold cavity because it was relatively thin and flimsy.

Referring to FIG. 7, there is a flowchart illustrating the steps of a preferred method 700 for making microplate 200, 300 and 400 using the thermally conductive material that is a metal. In the preferred embodiment, the microplate 200, 300 and 400 can be made from a metal including, for example, zinc, aluminum, magnesium and copper.

To manufacture the microplate 200, 300 and 400 the metal is heated and melted (step 702) and then injected (step 704) into a mold cavity (e.g., die cast) of a machine. The mold cavity includes sections shaped to form the microplate 200, 300 and 400. The machine then cools (step 706) the injected melted metal to create the microplate 200, 300 and 400. Finally, the microplate 200, 300 and 400 is removed (step 708) from the machine. Metal plates can also be manufactured by other known techniques such as metal particle injection molding (MIM), thixotropic or semi-solid processing techniques.

Another advantage of the present invention is that a microplate 200 and 300 with a large number of wells 204 and 304 (e.g., 1536 wells) having shared walls 206 and 306 is easier to manufacture than the traditional microplate that has 1536 wells with very thin unshared walls. Because, it is very difficult to mold the thin unshared walls that make-up each of the 1536 wells in the traditional microplate 100 without a large reduction of well volume.

Referring to FIG. 8, there is a flowchart illustrating the steps of a preferred method 800 for using the microplate 200, 300 and 400. Although the microplate 200, 300 and 400 of the present invention is described as being used in a PCR process, it should be understood that the microplate 200, 300 and 400 can be used in any process that can use a rigid microplate 200, 300 and 400.

Beginning at step 802, the scientist or robotic handling system places the microplate 200, 300, 400 into the thermocycler 500. The robotic handling system can handle the microplate 200, 300 and 400 if the microplate 200, 300 and 400 has a correctly sized footprint. Prior to placing the microplate 200, 300 and 400 into the thermocycler 500, the scientist can deposit a small quantity of genetic material and a solution of reactants into each well 204, 304 and 404 of the microplate 200, 300 and 400. And, then the scientist if need be can place a sealing film, mineral oil, wax or some other type of seal over the microplate 200, 300 and 400 to help prevent the evaporation of the contents within the wells 204, 304 and 404.

At step 804, the thermocycler 500 operates and cycles the temperature of contents within the wells 204, 304 and 404 of the microplate 200, 300 and 400 in accordance with the PCR process. For instance, the thermocycler 500 can cycle the temperature of the contents within the wells 204, 304 and 404 from 95° C. to 55° C. to 72° C. some thirty times during the PCR process.

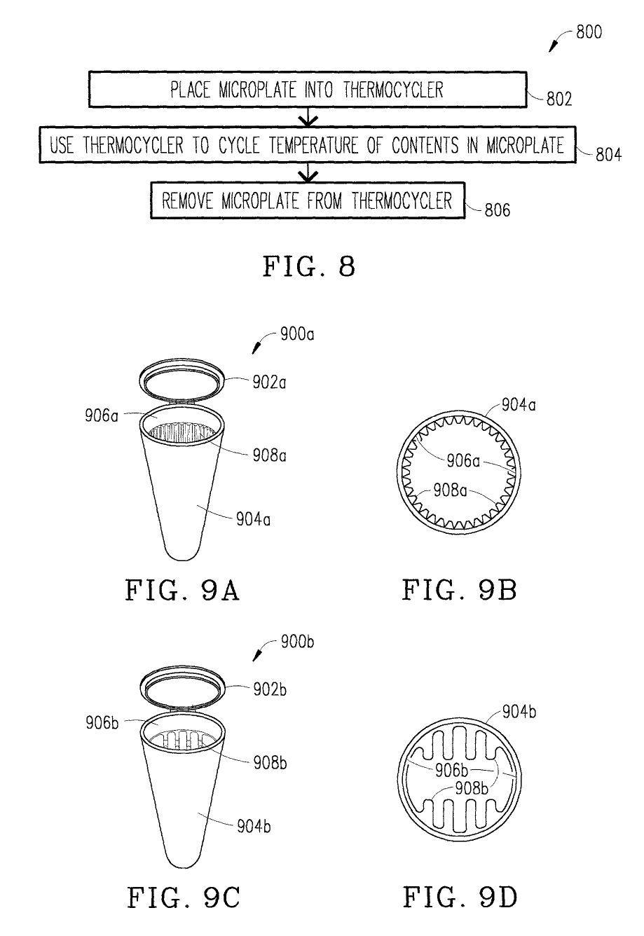

Lastly at step 806, the scientist or robotic handling system removes the microplate 200, 300 and 400 from the thermocycler 500. Again, the thermally conductive material (e.g., thermally conductive plastic or metal) used to make the microplate 200, 300 and 400 enhances the mechanical properties of microplate 200, 300 and 400 which makes it rigid and easier to remove the microplate 200, 300 and 400 from the thermocycler 500. This is a marked improvement over the traditional microplate 100 that had a tendency to deform and stick to the thermocycler 500 which made it difficult for the scientist or robot handling system to remove the traditional microplate 100 from the thermocycler 500. Referring to FIGS. 9A through 9D, there are illustrated a perspective view and a cross-sectional top view of two embodiments of a tube 900a and 900b in accordance with the present invention. Like the microplate 200, 300 and 400, the tube 900a and 900b is manufactured from a thermally conductive material that is a polymer (e.g., polypropylene, LCP . . . ) mixed with one or more thermally conductive additives (e.g., carbon fiber, metals, ceramic (boron nitride) . . . ). Or, the tube 900a and 900b is manufactured from a thermally conductive material that is a metal (e.g., aluminum, zinc . . . ). To avoid repetition, the different types of thermally conductive materials that can be used to make the tube 900a and 900b are not described in detail below since they are the same thermally conductive materials used to make the microplate 200, 300 and 400.

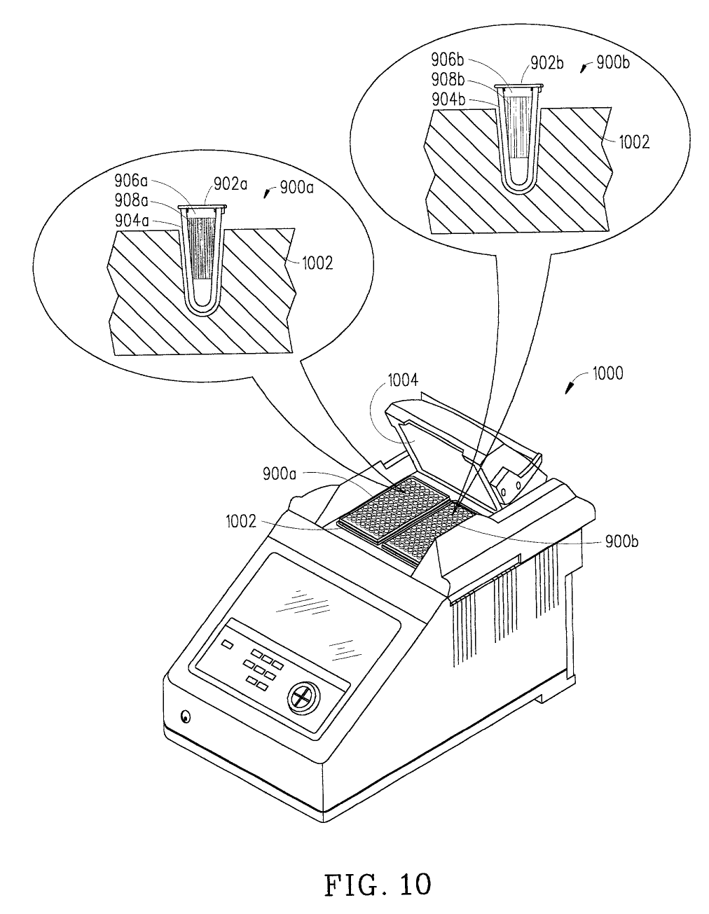

As illustrated, the tube 900a and 900b has a cap 902a and 902b that can be used to cover a well 904a and 904b. Each well 904a and 904b has an inner wall 906a and 906b that has a series of protruding heat transfer fins 908a and 908b (optional). The optional heat transfer fins 908a and 908b that extend out from the inner wall 906a and 906b function to increase the surface area within the well 904a and 904b . The additional surface area within the well 904a and 904b caused by the heat transfer fins 908a and 908b enables a thermocycler 1000 (see FIG. 10) to quickly cycle the temperature of a solution located within the well 904a and 904b.

The thermally conductive tube 900a and 900b with or without the heat transfer fins 908a and 908b is a marked improvement over traditional tubes. The traditional tubes do not have heat transfer fins because they are made from a polymer which is a relatively “poor” conductor of heat. If the traditional tubes had heat transfer fins they would actually slow down thermal conduction by acting to limit the useful surface area to transfer heat to and from the solution in the wells. In other words, the traditional tube does not have heat transfer fins because the heat transfer fins which are made from a polymer act as insulation which is just the opposite result one wants when they are using a thermocycler 1000 to heat and cool a solution in the well.

It should be understood that heat transfer fins 908a and 908b or similar fins could be added to the wells in microplates 200, 300 or 400. In practice, the microplate 200, 300 and 400 with such heat transfer fins 908a and 908b would have a relatively small number of wells such as 96 wells.

Referring to FIG. 10, there is a perspective view of an exemplary thermocycler 1000 capable of heating and cooling one or more tubes 900a and 900b. In accordance with the PCR process, a small quantity of genetic material and a solution of reactants are deposited within the well 904a and 904b of the tube 900a and 900b. The cap 902a and 902b then covers the well 904a and 904b to help prevent the evaporation of the contents within the well 904a and 904b. Thereafter, the tube 900a and 900b is placed in the thermocycler 1000 (e.g., GeneAmp® PCR System 9700) which operates to cycle the temperature of the contents within the well 904a and 904b.

As illustrated, tube 900a and 900b is positioned onto a metal heating fixture 1002 of the thermocycler 1000. The metal heating fixture 1002 can have a series of cavities each of which are shaped to closely conform to the exterior portion of the well 904a and 904b in tube 900a and 900b (see enlarged cross-sectional side views of the metal heating fixture 1002 and tubes 900a and 900b). The thermocycler 1000 also has a heated top plate 1004 (shown in the open position) that tightly clamps the tube 900a and 900b onto the metal heating fixture 1002 before the thermocycler 1000 repeatedly heats and cools the contents within the tube 900a and 900b. For instance, the thermocycler 1000 can cycle the temperature of the contents within the well 904a and 904b from 95° C. to 55° C. to 72° C. some thirty times during the PCR process. Again, the additional surface area within the well 904a and 904b caused by the heat transfer fins 908a and 908b enables the thermocycler 1000 to quickly cycle the temperature of a solution located within the well 904a and 904b.

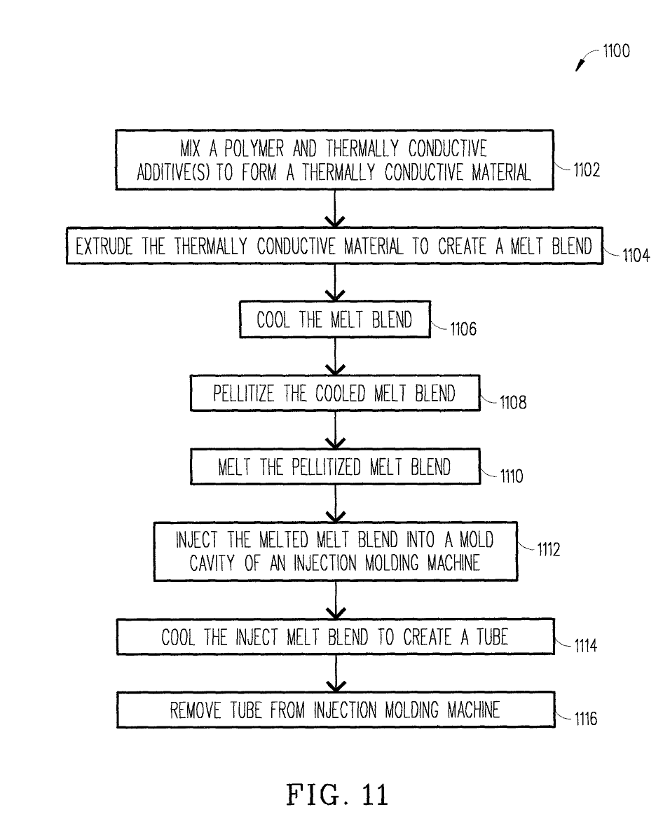

Referring to FIG. 11, there is a flowchart illustrating the steps of a preferred method 1100 for making tube 900a and 900b using the thermally conductive material that is a polymer mixed with one or more thermally conductive additives. The tube can be manufactured by mixing (step 1102) a polymer (e.g., crystalline polymer) and one or more thermally conductive additives to form a thermally conductive material. In the preferred embodiment, tube 900a and 900b is made from polymer such as polypropylene and a thermally conductive additive such as carbon fiber, metal, ceramic (boron nitride) . . .

The next step in manufacturing the tube 900a and 900b includes extruding (step 1104) the polymer that is mixed with one or more thermally conductive additives to create a melt blend. In particular, the polymer and thermally conductive additive(s) can be fed into a twin-screw extruder with the help of a gravimetric feeder to create a well-dispersed melt blend. The extruded melt blend is then run through a water bath and cooled (step 1106) before being pelletized (step 1108) and dried. The pelletized melt blend is heated and melted (step 1110) by an injection molding machine which then injects (step 1112) the melt blend into a mold cavity of the injection molding machine. The mold cavity includes sections shaped to form the tube 900a and 900b. The injection molding machine then cools (step 1114) the injected melt blend to create the tube 900a and 900b. Finally, the tube 900a and 900b is removed (step 1116) from the injection molding machine.

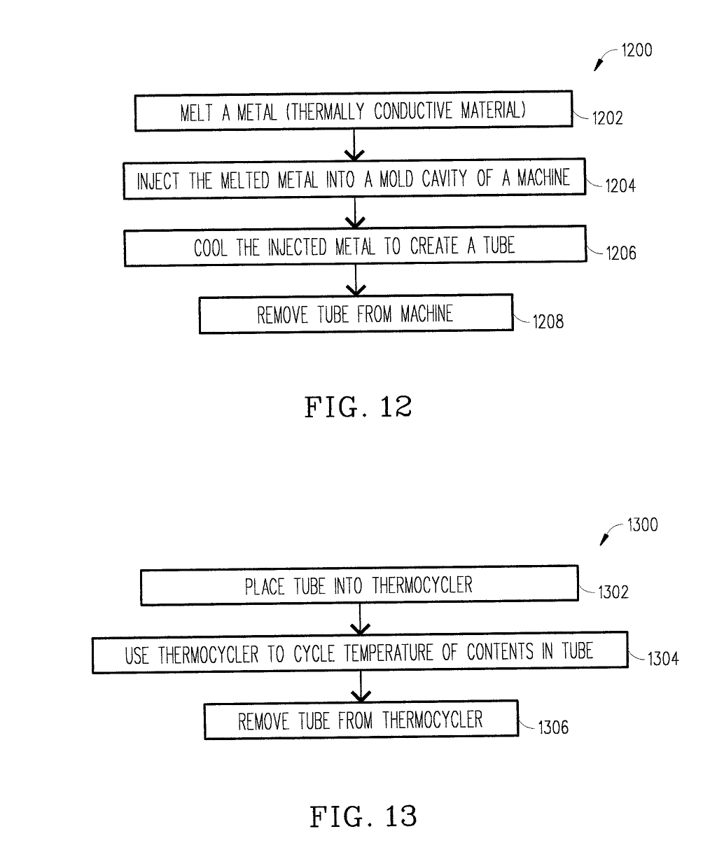

Referring to FIG. 12, there is a flowchart illustrating the steps of a preferred method 1200 for making tube 900a and 900b using the thermally conductive material that is a metal. In the preferred embodiment, the tube 900a and 900b is made from a metal including, for example, zinc, aluminum, magnesium and copper.

To manufacture the tube 900a and 900b the metal is heated and melted (step 1202) and then injected (step 1204) into a mold cavity (e.g., die cast) of a machine. The mold cavity includes sections shaped to form the tube 900a and 900b. The machine then cools (step 1206) the injected melted metal to create the tube 900a and 900b. Finally, the tube 900a and 900b is removed (step 1208) from the machine.

Although only two configurations of heat transfer fins 908a and 908b in tube 900a and 900b have been shown, it should be understood that the present invention is not limited to these configurations. Instead, the tubes of the present invention can have heat transfer fins with a wide variety of configurations so long as the heat transfer fins effectively increase the surface area within the well. Again, the heat transfer fins increase the surface area within a well which enables a thermocycler to more quickly cycle the temperature of a solution located within the well when compared to the traditional tubes and tubes 900a and 900b without the heat transfer fins 908a and 908b.

Referring to FIG. 13, there is a flowchart illustrating the steps of a preferred method 1300 for using the tube 900a and 900b. Although the tube 900a and 900b of the present invention is described as being used in a PCR process, it should be understood that the tube 900a and 900b can be used in any process that can use a rigid tube 900a and 900b.

Beginning at step 1302, the scientist places the tube 900a and 900b into the thermocycler 1000. Prior to placing the tube 900a and 900b into the thermocycler 1000, the scientist can deposit a small quantity of genetic material and a solution of reactants into the well 904a and 904b of the tube 900a and 900b. And, then the scientist can move the cover 902a and 902b over the well 904a and 904b to help prevent the evaporation of the contents within the well 904a and 904b.

At step 1304, the thermocycler 1000 operates and cycles the temperature of contents within the well 904a and 904b of the tube 900a and 900b in accordance with the PCR process. For instance, the thermocycler 1000 can cycle the temperature of the contents within the well 904a and 904b from 95° C. to 55° C. to 72° C. some thirty times during the PCR process. Lastly at step 1306, the scientist removes the tube 900a and 900b from the thermocycler 1000.

Although several embodiments of the present invention has been illustrated in the accompanying Drawings and described in the foregoing Detailed Description, it should be understood that the invention is not limited to the embodiments disclosed, but is capable of numerous rearrangements, modifications and substitutions without departing from the spirit of the invention as set forth and defined by the following claims.

What is claimed is:

1. A microplate, comprising:
a frame including a plurality of wells formed therein, said frame is manufactured from a thermally conductive material that enables the wells to have relatively rigid walls which makes it easier to handle said frame.

2. The microplate of claim 1, wherein said frame can be easily removed from a thermocycler.

3. The microplate of claim 1, wherein said frame can be easily handled by a robotic handling system.

4. The microplate of claim 1, wherein each well an exterior with a conical shaped bottom or a flat shaped bottom.

5. The microplate of claim 1, wherein each well shares a wall with adjacent wells.

6. The microplate of claim 1, wherein said frame includes a skirt connected to one or more wells by one or more ribs.

7. The microplate of claim 1, wherein said thermally conductive material is a mixture of a polymer and at least one thermally conductive additive.

8. The microplate of claim 1, wherein said thermally conductive material is a metal.

9. The microplate of claim 1, wherein said thermally conductive material has a thermal conductivity that is greater than 1.0 W/mk.

10. The microplate of claim 1, wherein said thermally conductive material has a thermal conductivity that is greater than 5.0 W/mk.

11. The microplate of claim 1, wherein said thermally conductive material has a thermal conductivity that is greater than 50.0 W/mk.

12. A microplate manufactured in such a way so as to improve the ability to properly carry out a polymerase chain reaction process, said microplate comprising:
a frame including a plurality of wells formed therein, said frame is manufactured from a thermally conductive material that enables the wells to have relatively thick walls which makes it easier to remove said frame from a thermocycler.

13. The microplate of claim 12, wherein each well an exterior with a conical shaped bottom or a flat shaped bottom.

14. The microplate of claim 12, wherein each well shares a wall with adjacent wells.

15. The microplate of claim 12, wherein said frame includes a skirt connected to one or more wells by one or more ribs.

16. The microplate of claim 12, wherein said thermally conductive material is a mixture of a polymer and at least one thermally conductive additive.

17. The microplate of claim 16, wherein said at least one thermally conductive additive has a thermal conductivity greater than a thermal conductivity of said polymer.

18. The microplate of claim 16, wherein said polymer can be a crystalline polymer.

19. The microplate of claim 16, wherein said at least one thermally conductive additive is carbon fiber, metal or ceramic.

20. The microplate of claim 12, wherein said thermally conductive material is a metal.

21. The microplate of claim 12, wherein said thermally conductive material has a thermal conductivity that is greater than 1.0 W/mk.

22. The microplate of claim 12, wherein said thermally conductive material has a thermal conductivity that is greater than 5.0 W/mk.

23. The microplate of claim 12, wherein said thermally conductive material has a thermal conductivity that is greater than 50.0 W/mk.

24. A method for making a microplate, said method comprising the steps of:
mixing a polymer and at least one thermally conductive additive;
extruding the mixed polymer and the at least one thermally conductive additive to create a melt blend;
cooling said extruded melt blend;
pelletizing said cooled melt blend;
melting said pelletized melt blend;
injecting said melted blend into a mold cavity of an injection molding machine, said mold cavity includes sections shaped to form said microplate;
cooling the injected melt blend to create said microplate; and
removing said microplate from the injection molding machine, wherein said microplate includes a plurality of wells.

25. The method of claim 24, wherein said microplate includes a skirt connected to one or more wells by one or more ribs.

26. The method of claim 24, wherein each well has an exterior with a conical shaped bottom or a flat shaped bottom.

27. The method of claim 24, wherein said at least one thermally conductive additive has a thermal conductivity greater than a thermal conductivity of said polymer.

28. The method of claim 24, wherein said polymer is a crystalline polymer.

29. The method of claim 24, wherein said at least one thermally conductive additive is carbon fiber, metal or ceramic.

30. A method for making a microplate, said method comprising the steps of:
melting a thermally conductive material;
injecting said melted thermally conductive material into a mold cavity of a machine, said mold cavity includes sections shaped to form said microplate;
cooling the injected thermally conductive material to create said microplate; and
removing said microplate from the machine, wherein said microplate includes a plurality of wells.

31. The method of claim 30, wherein said microplate includes a skirt connected to one or more wells by one or more ribs.

32. The method of claim 31, wherein each well has an exterior with a conical shaped bottom or a flat shaped bottom.

33. The method of claim 30, wherein said thermally conductive material is a metal.

34. A method for using a microplate, said method comprising the steps of:
placing the microplate into a thermocycler;
operating the thermocycler so as to cycle the temperature of a solution within one or more wells in said microplate; and
removing the microplate from the thermocycler, wherein said microplate is manufactured from a thermally conductive material that enables the wells to have relatively thick walls which makes it easier to remove said microplate from the thermocycler.

35. The method of claim 34, wherein said microplate includes a skirt connected to one or more wells by one or more ribs.

36. The method of claim 34, wherein each well has an exterior with a conical shaped bottom or a flat shaped bottom.

37. The method of claim 34, wherein said thermally conductive material is a mixture of a polymer and at least one thermally conductive additive.

38. The method of claim 37, wherein said at least one thermally conductive additive has a thermal conductivity greater than a thermal conductivity of said polymer.

39. The method of claim 37, wherein said polymer can be a crystalline polymer.

40. The method of claim 37, wherein said at least one thermally conductive additive is carbon fiber, metal or ceramic.

41. The method of claim 34, wherein said thermally conductive material is a metal.

42. The method of claim 34, wherein said thermally conductive material has a thermal conductivity that is greater than 1.0 W/mk.

43. The method of claim 34, wherein said thermally conductive material has a thermal conductivity that is greater than 5.0 W/mk.

44. The method of claim 34, wherein said thermally conductive material has a thermal conductivity that is greater than 50.0 W/mk.

45. A tube manufactured in such a way so as to improve the ability to properly carry out a polymerase chain reaction process, said tube comprising:
a well manufactured from a thermally conductive material that enables the well to have a relatively rigid wall.

46. The tube of claim 45, wherein said well further includes a plurality of protruding heat transfer fins which increases the surface area within the well which in turn enables a thermocycler to quickly cycle the temperature of a solution within the well.

47. The tube of claim 45, further includes a cap that covers the well.

48. The tube of claim 45, wherein said thermally conductive material is a mixture of a polymer and at least one thermally conductive additive.

49. The tube of claim 48, wherein said at least one thermally conductive additive has a thermal conductivity greater than a thermal conductivity of said polymer.

50. The tube of claim 48, wherein said polymer can be a crystalline polymer.

51. The tube of claim 48, wherein said at least one thermally conductive additive is carbon fiber, metal or ceramic.

52. The tube of claim 45, wherein said thermally conductive material is a metal.

53. The tube of claim 45, wherein said thermally conductive material has a thermal conductivity that is greater than 1.0 W/mk.

54. The tube of claim 45, wherein said thermally conductive material has a thermal conductivity that is greater than 5.0 W/mk.

55. The tube of claim 45, wherein said thermally conductive material has a thermal conductivity that is greater than 50.0 W/mk.

56. A method for making a tube, said method comprising the steps of:
melting a thermally conductive material;
injecting said melted thermally conductive material into a mold cavity of an injection molding machine, said mold cavity includes sections shaped to form said tube;
cooling the injected thermally conductive material to create said tube; and
removing said tube from the injection molding machine, wherein said tube includes a well with an inner wall having a plurality of heat transfer fins extending therefrom.

57. The method of claim 56, wherein said thermally conductive material is a mixture of a polymer and at least one thermally conductive additive.

58. The method of claim 56, wherein said thermally conductive material is a metal.

59. A method for using a tube, said method comprising the steps of:
placing said tube into a thermocycler, said tube is made from a thermally conductive material and includes a well with an inner wall having a plurality of heat transfer fins extending therefrom;
operating the thermocycler so as to cycle the temperature of contents within the well of said tube; and
removing said tube from the thermocycler.

60. The method of claim 59, wherein said thermally conductive material is a mixture of a polymer and at least one thermally conductive additive.

61. The method of claim 59, wherein said thermally conductive material is a metal.
